(19) United States
(12) Patent Application Publication
(10) Pub. No.: US 2006/0040339 A1
(43) Pub. Date: Feb. 23, 2006

(54) Method of constructing a calibration curve in microorganism assay

(76) Inventors: Naoki Fukui, Tsukuba-shi (JP); Megumi Akamatsu, Tsukuba-shi (JP); Seiichiro Miyahara, Tsukuba-shi (JP); Chiaki Okumura, Tsukuba-shi (JP); Toshimi Murata, Brookline, MA (US); Yoshihisa Amano, Riverside, CA (US)

Correspondence Address: BIRCH STEWART KOLASCH & BIRCH, PO BOX 747, FALLS CHURCH, VA, 22040-0747, US

(21) Appl. No.: 10/919,290

(22) Filed: Aug. 17, 2004

(51) Int. Cl. G06F 19/00; C12Q 1/04

(52) U.S. Cl. 435/34; 702/19

(57) ABSTRACT

A method of constructing a calibration curve in a determination of microbial counts is provided. An immunoassay of the present invention is characterized in that samples of a specimen material are prepared so that microbial counts therein are altered.

### FILED OF THE INVENTION

The present invention relates to a method of constructing a calibration curve in the course of a process for determining the microbial count in a specimen material. Specifically, the invention relates to a method of constructing a calibration curve which comprises a step of preparing samples suitable for constructing a calibration curve, and to an apparatus for determination of microbial counts which comprises said method.

### BACKGROUND OF THE INVENTION

Hygiene control of foodstuffs such as meat, vegetables, and dairy products demands the determination of microbial counts therein. To meet the demand, various methods of determining microbial counts comprising culturing a foodstuff specimen have been proposed such as a method in which the respiration rate of the microbes is measured (Japanese Patent Publication (Kokai) No. S56-140898 (1981)), a method in which the decrease in the amount of dissolved oxygen due to respiration of the microbes is measured (Japanese Patent Publication (Kokai) No. S63-15150 (1988)), a method in which the impedance of the solution to evaluate metabolites from the microbes is measured (Impedance method), and a method in which ATP is specifically measured by the luciferin-luciferase reaction (ATP-bioluminescence method, ATP method).

In all of the methods, a calibration curve must be constructed to determine the microbial counts in a foodstuff specimen.

### SUMMARY OF THE INVENTION

In order to obtain calibration curves that yield highly accurate analysis of corresponding foodstuffs, it is desirable to use foodstuff specimens, each of which has microbial counts varied over a wide range. To this end, various specimens are selected from the foodstuffs to prepare samples having widely varied microbial counts, and foodstuff specimens are repeatedly selected until a calibration curve with a high coefficient of correlation is obtained. Since it is general that untreated normal foodstuffs are usually uniform in the microbial count, only a combination of particular microbes is typically added to the foodstuff to expand the range of the microbial counts, thereby constructing a calibration curve. However, typical methods in which microbes are added to the foodstuff is not suitable for such methods of determining the microbial count as a method in which the microbial count in a foodstuff is calculated based on metabolites from the microbes, for example, a method comprising measuring dissolved oxygen or the Impedance method, because the proliferation rate or metabolic rate of microbes is influenced by the conditions specific to the foodstuff including the community structure of existing microbes, nutrients in the foodstuff, and the presence or the absence of proliferation-inhibiting ingredients such as spices or antiseptics.

Under such circumstances, the present inventors conducted assiduous studies and found a simple and convenient approach for bringing the microbial count in a foodstuff within a desired range, thereby accomplishing the method of the present invention for constructing a calibration curve.

Thus, the present invention relates to method of constructing a calibration curve in the course of a process for determining the microbial count in a specimen material, the method comprising the steps of:
1) preparing samples of the specimen material that have altered microbial counts in a number sufficient for constructing a calibration curve,
2) measuring a predetermined parameter for each of the samples obtained in Step 1, and, at the same time, determining quantitatively the microbial count in each of the corresponding samples, and
3) correlating the measured values for the predetermined parameter with the microbial counts measured by the quantitative determination, thereby constructing a calibration curve.

In another embodiment, the present invention relates to an apparatus for determination of microbial counts comprising, or for conducting, a method of constructing a calibration curve according to the present invention.

In a further embodiment, the present invention relates to a process for determining microbial counts in a specimen material, which comprises a method of constructing a calibration curve according to the present invention, and an apparatus for determination of microbial counts comprising the process.

### BRIEF DESCRIPTION OF THE DRAWINGS

FIG. 1: A flowchart diagram illustrating a determination of microbial counts as carried out in a method of constructing a calibration curve according to the present invention which comprises DOX.

FIG. 2: A schematic diagram illustrating a method of constructing a calibration curve according to the present invention which comprises DOX.

FIG. 3: A flowchart diagram illustrating a method of constructing a calibration curve with DOX, wherein the method employs samples of a specimen material having altered microbial counts which are prepared by keeping the specimen material at ambient temperature or at a lower temperature to increase the microbial count, or by freezing the specimen material to decrease the microbial count.

FIG. 4: A flowchart diagram illustrating a method of constructing a calibration curve with DOX, wherein the method employs samples of a specimen material having altered microbial counts which are prepared by proliferating the microbes using a liquid material obtained by homogenizing the specimen material, and diluting the proliferated microbes as appropriate.

FIG. 5: A flowchart diagram illustrating a method of constructing a calibration curve with a DOX apparatus, wherein the method employs samples of a specimen material having altered microbial counts which are prepared by proliferating the microbes using a liquid material obtained by homogenizing the specimen material, diluting the proliferated microbes as appropriate, and then adding each of the diluted materials thus prepared to a homogenized liquid material of the specimen material.

FIG. 6: A flowchart diagram illustrating a method of constructing a calibration curve with DOX, wherein the method employs samples of a specimen material having altered microbial counts which are prepared, after a measurement is conducted in DOX using a liquid material obtained by homogenizing the specimen material, by removing the liquid out of DOX, and diluting said liquid as appropriate.

FIG. 7: A flowchart diagram illustrating a method of constructing a calibration curve with DOX, wherein the method employs samples of a specimen material having altered microbial counts which are prepared, after a measurement is conducted in DOX using a liquid material obtained by homogenizing the specimen material, by removing the liquid out of DOX, diluting said liquid as appropriate, and adding each of the diluted materials thus prepared to a homogenized liquid material of the specimen material.

FIG. 8: A protocol for calibration curve construction.

FIG. 9: Calibration curves constructed by various methods using beef as a foodstuff.

FIG. 10: Calibration curves constructed by various methods using chicken as a foodstuff.

### DETAILED DESCRIPTION OF THE PREFERRED EMBODIMENTS

The method of constructing a calibration curve according to the present invention comprises Step 1 comprising preparing samples of the specimen material that have altered microbial counts, Step 2 comprising measuring a predetermined parameter for each of the samples obtained in Step 1, and, at the same time, determining quantitatively the microbial count in each of the corresponding samples, and Step 3 comprising correlating the measured values for the predetermined parameter with the microbial counts measured by the quantitative determination, thereby constructing a calibration curve. Each step will be described in detail below.

Step 1

As used herein, the term “specimen material” refers to an object for which the microbial count will be determined, and specimen materials typically include, but not limited to, foodstuffs such as meat, vegetables, marine products, everyday dishes, and dairy products, as well as pharmaceuticals, and chemical products.

As used herein, the term “sample” means a material derived from an extract such as a liquid material obtained by homogenizing a specimen material via for example a stomacher or a homogenizer, and extracting the treated material.

Thus, the term “samples that have an altered microbial count” means materials, such as a liquid material obtained by homogenizing a specimen material, wherein the microbial count has been altered by a certain technique. The samples as defined above also includes a sample prepared by adding a material in which the microbial count has been altered by the following techniques to an extract such as a liquid material obtained by homogenizing a specimen material.

According to the present invention, techniques for altering the microbial count in a sample are described as follows.

As a first technique, a sample of a specimen material that has an altered microbial count can be prepared by keeping the specimen material at ambient temperature or at a lower temperature to increase the microbial count, or by freezing the specimen material to decrease the microbial count.

This technique provides samples having an ideal range of the microbial counts.

Specifically, in case that the specimen material is meat, the material may be stored at around 4° C. for about one day to one week, because the microbial count increases too much at ambient temperature, and adequately increases even at a lower temperature. In case that the specimen material is a common processed food, for example, a precooked everyday dish or a dairy product, it may be stored at ambient temperature. A specimen material having a high microbial count such as a liver may be frozen to decrease the microbial count. For example, an experiment that shows the microbial count in meat was decreased by an order of magnitude by freezing for one week, has been reported.

As a second technique, a sample of a specimen material that has an altered microbial count can be prepared by homogenizing the specimen material to give a liquid material, proliferating the microbes in the liquid material, and diluting the proliferated microbes as appropriate.

This technique is useful when it is difficult to increase the microbial count by the first technique.

As a procedure for proliferating microbes, the standard plate count method (the plate method) or a liquid culture method is usually used to increase the microbial count. In the plate method, for example, a liquid material obtained by homogenizing a specimen material is smeared on a Petri dish containing standard agar medium, and then incubated for 48 hours at 35° C. Microbes are then picked from predetermined colonies in such a way that they reflect the proportions of the types and the amounts of generated colonies, and serially diluted with a physiological saline to provide diluted materials, which represent samples having altered microbial counts.

This technique is suitable where the initial microbial count in the specimen material is low, because if the homogenized liquid material, to which the diluted material is added, already contains a large amount of microbes, the microbial count of the diluted material to be added may have as little significance as an error.

As a third technique, a sample of the specimen material that has an altered microbial count can be prepared by adding each of the diluted materials as prepared in the second technique to a liquid material obtained by homogenizing the specimen material.

The addition of said diluted materials to a liquid material obtained by homogenizing the specimen material, makes it possible to set up an environment for microbial proliferation which reflects the conditions specific to the specimen material, for examples, nutrients in the foodstuff, the presence or absence of proliferation-inhibiting ingredients such as spices or antiseptics.

Step 2

Step 2 comprises measuring a predetermined parameter for each of the samples prepared in Step 1, and at the same time, determining quantitatively the microbial count in each of the corresponding samples.

A predetermined parameter for the samples prepared in Step 1 may be usually measured by an oxygen electrode method wherein concentrations of dissolved oxygen are determined, or by the impedance method (“Yugai-Biseibutsu-Kannri-Gijutsu vol. 2/Seizou-Ryutsu-Kankyou-Ni-Okeru-Engineering-to-HACCP (Controltechnologies against hazardous microbes, vol. 2/Engineering and HACCP in production and distribution environments)”, p. 472).

An oxygen electrode method which may be used herein is described in detail in our PCT application WO0061791 (PCT/JP00/02305).

“Quantitative determination of the microbial count” refers to measurement of the actual microbial count, and is usually achieved using the standard plate count method (the plate method).

To determine quantitatively the microbial count using the plate method, a corresponding homogenized liquid material is diluted with a physiological saline to prepare usually three serial 10-fold diluted materials, which are then subjected to the plate method. Simultaneously, a predetermined parameter is also measured.

Step 3

Step 3 comprises correlating the measured values for the predetermined parameter with the microbial counts measured by the quantitative determination of the microbial count so as to construct a calibration curve.

It is preferable that the calibration curve has a correlation coefficient, r, of 0.75 or higher, and plots span 4 or more orders of magnitude within the range of the microbial counts from 10² to 10⁷ CFU/g specimen material (for example 10³ to 10⁶). The number of plots may be 10 to 50 as a measuring stick for success, and more numbers of plots are preferable.

A regression curve is calculated by least square methods using the results obtained by measuring the predetermined parameter and the results obtained by determining quantitatively the microbial counts, thereby constructing a calibration curve.

In Step 2 of the present invention, the predetermined parameter may be measured with a commonly used apparatus for determining the microbial count. Such apparatuses for determining the microbial count include an apparatus which comprises measuring carbon dioxide concentration to evaluate the respiration rate of the microbes, an apparatus which comprises measuring the decrease in the amount of dissolved oxygen due to respiration of the microbes (DOX manufactured by Daikin Environmental Lab.; hereinafter abbreviated as DOX), an apparatus which comprises measuring the impedance of the solution to evaluate metabolites from the microbes, and an apparatus which comprises specifically measuring ATP by making use of the luciferin-luciferase reaction.

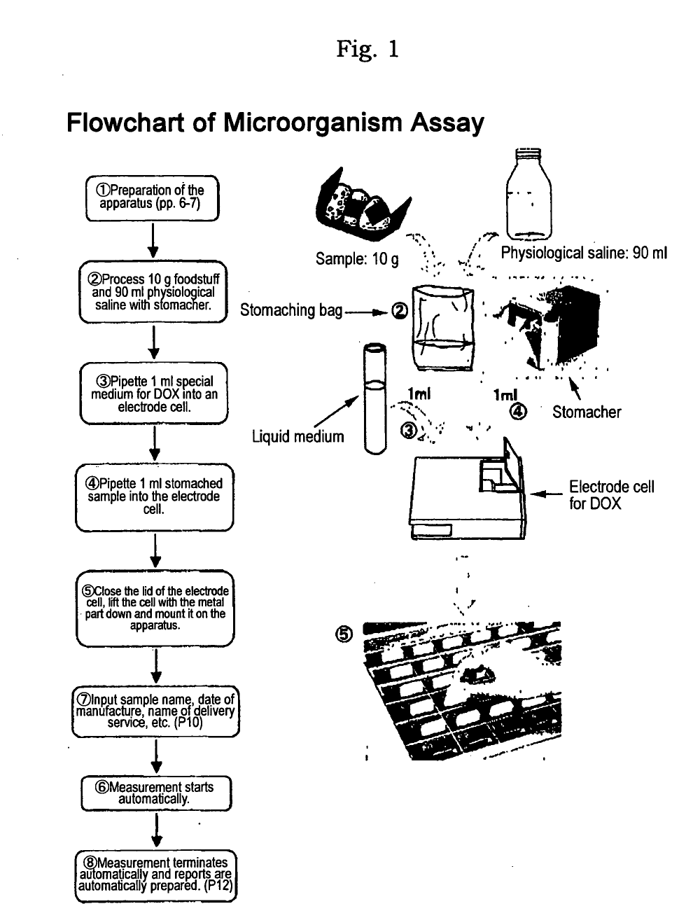

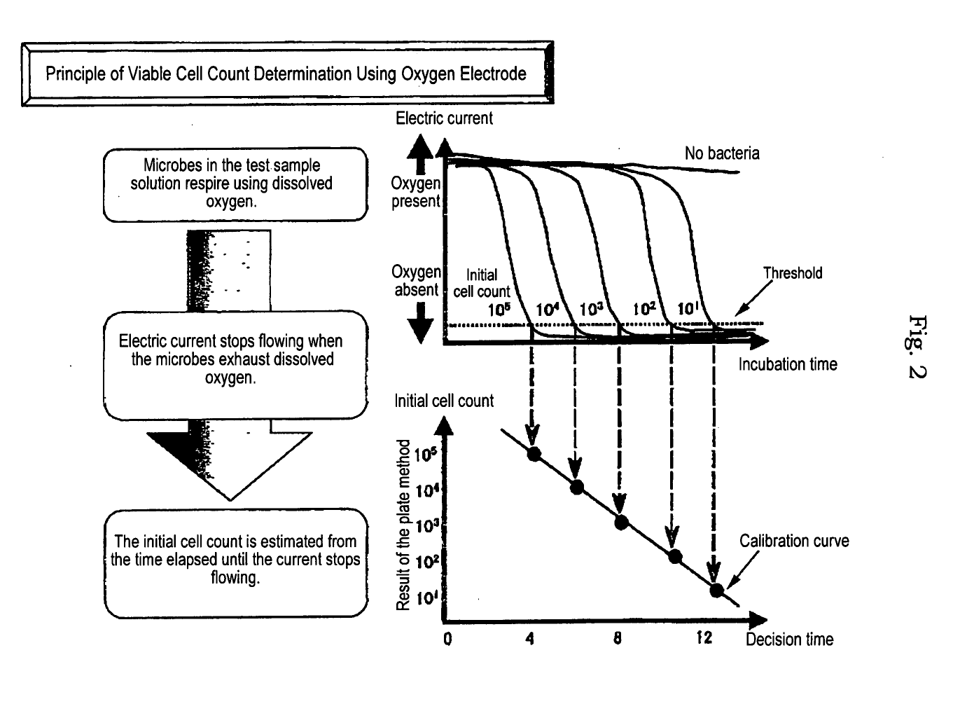

Regarding an embodiment of a method of constructing a calibration curve according to the present invention wherein an apparatus (DOX) is used which comprises measuring the decrease in the amount of dissolved oxygen due to respiration of the microbes to determine the microbial count, a flowchart for such determination of microbial counts may be described in FIG. 1, and the method of constructing a calibration curve may be schematically described in FIG. 2.

Specifically, a method of constructing a calibration curve according to the present invention which comprises DOX is carried out as follows.

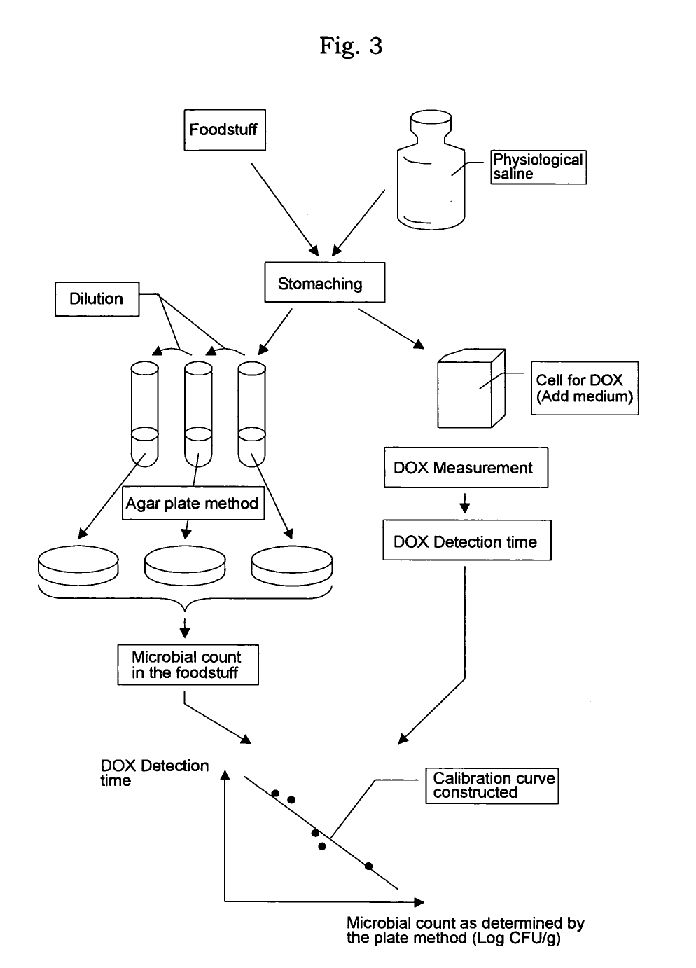

In an embodiment wherein the samples of the specimen material that have altered microbial counts are prepared in Step 1 by keeping the specimen material at ambient temperature or at a lower temperature to increase the microbial count, or by freezing the specimen material to decrease the microbial count, the microbial count are determined according to the flowchart shown in FIG. 3.

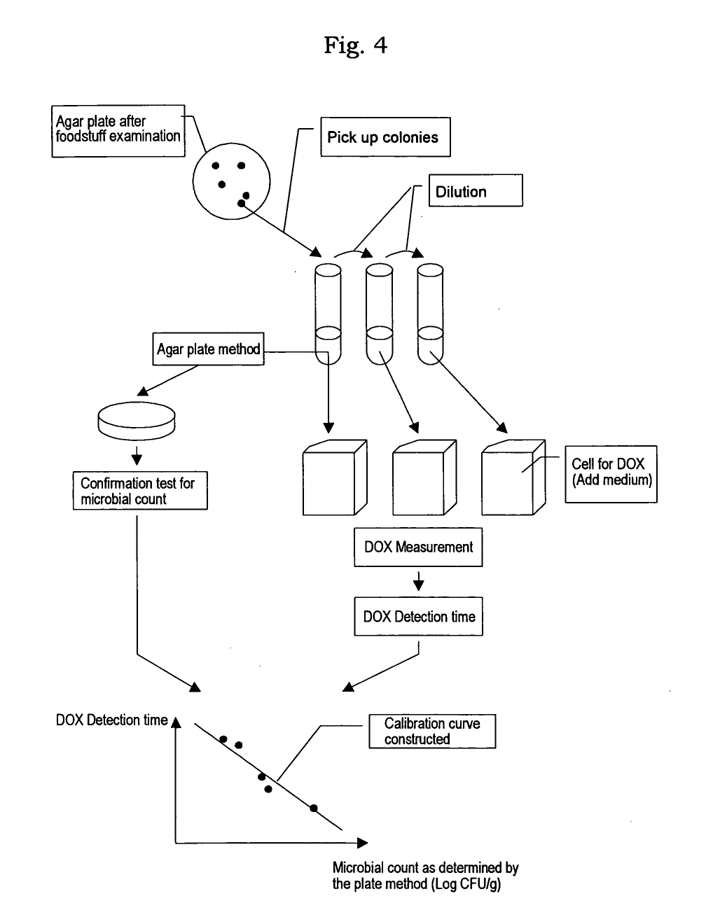

Likewise, in an embodiment wherein the samples of the specimen material that have altered microbial counts are prepared in Step 1 by proliferating the microbes with the plate method using a liquid material obtained by homogenizing the specimen material, and diluting the proliferated microbes as appropriate, the microbial count can be determined according to the flowchart shown in FIG. 4.

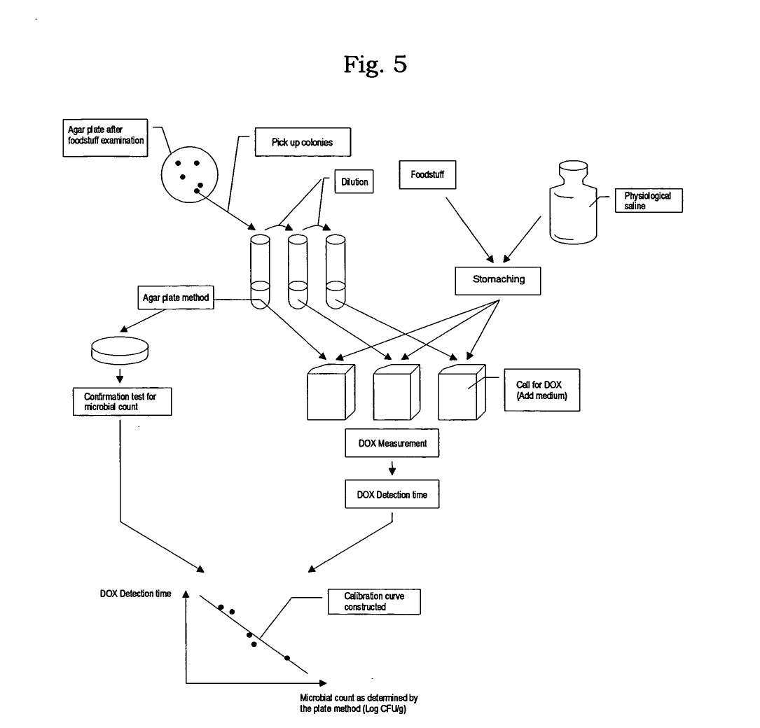

Furthermore, in an embodiment wherein, in Step 1, the samples of the specimen material that have altered microbial counts are prepared by proliferating the microbes with the plate method using a liquid material obtained by homogenizing the specimen material, diluting the proliferated microbes as appropriate, and adding each of the diluted materials thus prepared to a homogenized liquid material of the specimen material, the microbial count can be determined according to the flowchart shown in FIG. 5.

Additionally, in an embodiment wherein an apparatus which comprises measuring the decrease in the amount of dissolved oxygen due to respiration of the microbes to determine the microbial count, or an apparatus which comprises measuring the impedance of the solution to evaluate metabolites from the microbes, thus determining the microbial count, is used, the preparation, in step 1, of samples of the specimen material that have altered microbial counts may be carried out by the apparatus itself.

That is, in preparing samples by diluting the proliferated microbes as appropriate, the proliferation of microbes is carried out by the apparatus.

By using DOX, a method of constructing a calibration curve according to the present invention in which proliferation of microbes is carried out by the apparatus is conducted as follows.

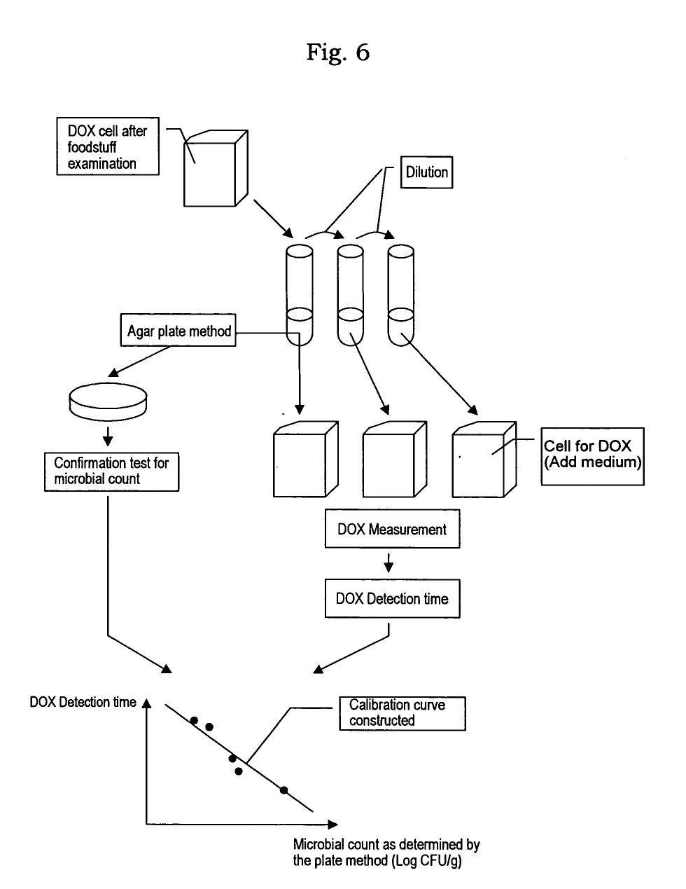

In Step 1, after a measurement is conducted in DOX using a liquid material obtained by homogenizing a specimen material, the liquid is removed out of DOX. Although this DOX measurement doesn't provide any quantified results, a rough estimate of the microbial count may be obtained, and in case that, for example, the microbial count proved to be zero, one can omit the subsequent steps. The liquid obtained is diluted as appropriate to prepare samples of the specimen material that have altered microbial counts. Such procedure can be adopted because, in DOX, microbes are allowed to proliferate in an electrode cell with a liquid medium. In this case, the microbial count can be determined according to the flowchart shown in FIG. 6. In this procedure, samples can be prepared in minimum one day. According to this procedure, the mixing ratio of microbes can be made more closely resemble to the actual community structure of microbes, compared to the procedure in which the microbial count is increased by the plate method.

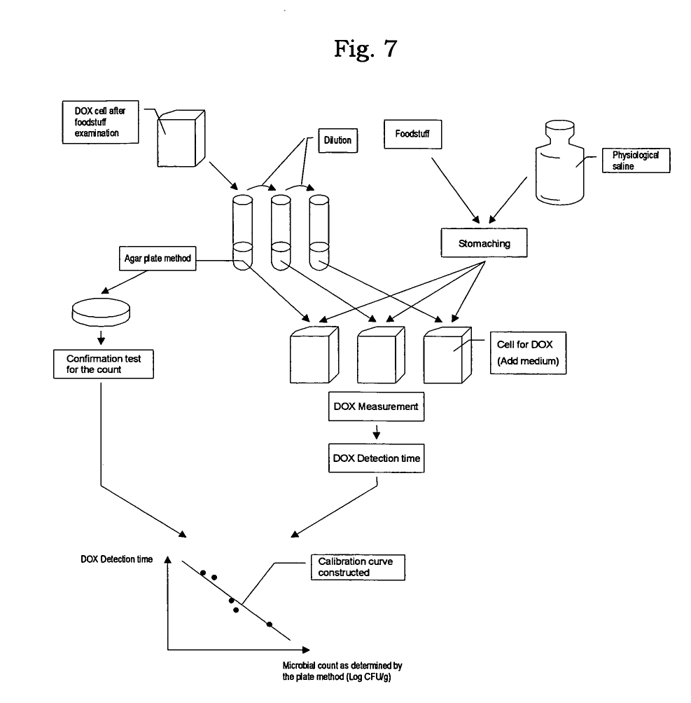

Likewise, in an embodiment wherein, in Step 1, after a measurement is carried out in DOX using a liquid material obtained by homogenizing a specimen material, the liquid is removed out of DOX, diluted as appropriate, and adding each of the diluted materials thus prepared to a homogenized liquid material of the specimen material to prepare samples of the specimen material that have altered microbial counts, the microbial count can be determined according to the flowchart shown in FIG. 7. Also in this procedure, samples can be prepared in minimum one day. According to this procedure, the mixing ratio of microbes can be made more closely resemble to the actual community structure of microbes, compared to the procedure in which the microbial count is increased by the plate method.

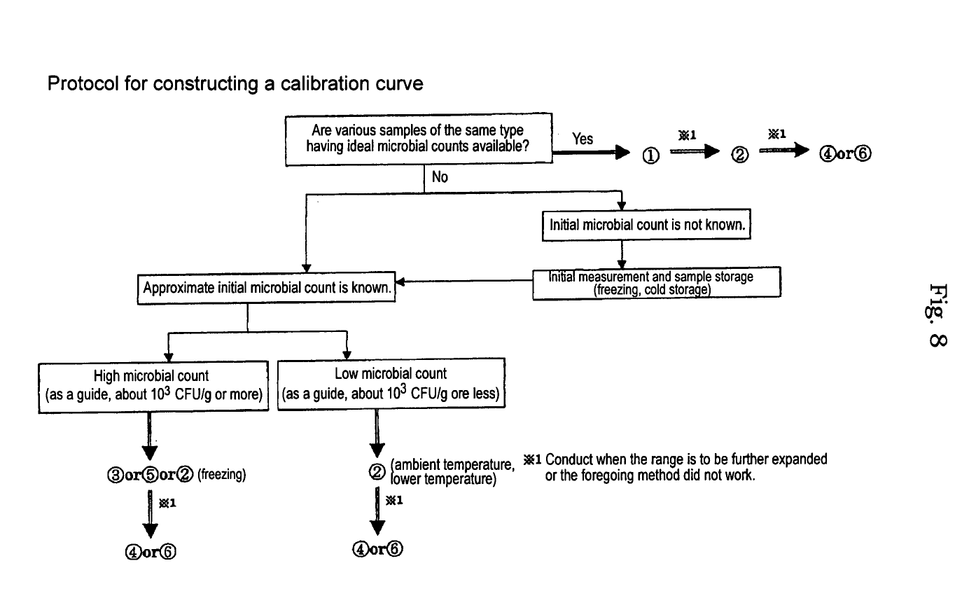

A practical protocol for constructing a calibration curve for a specimen material is shown in FIG. 8. In FIG. 8, (I) denotes the conventionally-used standard plate count method (the plate method) in which various samples of the same type are used. In this method, at least 3 different samples are used to construct a calibration curve. (II) denotes a measurement according to the flowchart shown in FIG. 3, in which the specimen material is stored under predetermined conditions to increase or decrease the microbial count. (III) denotes a measurement according to the flowchart shown in FIG. 4, in which microbes are proliferated by the plate method using a liquid material obtained by homogenizing the specimen material, and the proliferated microbes are diluted as appropriate. (IV) denotes a measurement according to the flowchart shown in FIG. 5, in which the diluted materials prepared in (III) are added to a homogenized liquid material of the specimen material. (V) denotes a measurement according to the flowchart shown in FIG. 6, in which microbes are proliferated in DOX using a liquid material obtained by homogenizing the specimen material, and diluted as appropriate. (VI) denotes a measurement according to the flowchart shown in FIG. 7, in which the diluted materials obtained by appropriate dilution in (V) are added to a homogenized liquid material of the specimen material.

In another aspect, the present invention provides an apparatus for determination of microbial counts comprising, or for conducting, a method of constructing a calibration curve according to the present invention.

Examples of an apparatus for determination of microbial counts comprising a method of the present invention are an apparatus which determines the microbial count by measuring the respiration rate of the microbes, an apparatus which determines the microbial count by measuring the decrease in the amount of dissolved oxygen due to respiration of the microbes, an apparatus which determines the microbial count by measuring the impedance of the solution to evaluate metabolites from the microbes, and an apparatus which specifically measures ATP by making use of the luciferin-luciferase reaction.

In another aspect, the present invention provides a process for determining microbial counts in a specimen material, which comprises a method of constructing a calibration curve in the course of a process for determining the microbial count in a specimen material according to the present invention, and an apparatus for determination of microbial counts comprising the process according to the invention.

### EXAMPLES

The present invention is illustrated in more detail in the following examples, to which the invention is not limited in any respect.

### Example 1

In these examples, each calibration curve was constructed using beef and chicken as a foodstuff. Therein, an apparatus which comprises measuring the decrease in the amount of dissolved oxygen due to respiration of the microbes to determine the microbial count (DOX; Daikin Environmental Lab., LTD.) was used as an apparatus for determining the microbial count.

Construction Method (I)

Construction Method (I) is a conventionally-used common method of constructing a calibration curve in which various untreated samples of the same type are used.

Ten gram of a foodstuff and 90 ml of physiological saline were placed into a specimen bag for homogenizer (containing 0.85% sodium chloride solution) and homogenized for one minute with a Pro-Media SH-011 (Elmex).

In order to measure the decrease in the amount of dissolved oxygen due to respiration of the microbes, 1 ml of the homogenized liquid material thus obtained and 1 ml of a special medium for DOX (yeast extract: 2.5 g, peptone: 5.0 g, and glucose: 1.0 g per 1 L liquid medium) were injected into an electrode-equipped cell, and the concentration of dissolved oxygen was measured. In order to determine quantitatively the microbial counts using the plate method, 1 ml of the homogenized liquid material obtained by the homogenization step, 1 ml of 10-fold dilution thereof, and 1 ml of 100-fold dilution thereof were respectively distributed into Petri dishes, and an agar medium was then poured. After culturing 48 hours at 35° C., the microbial counts were determined.

This operation was carried out with 6 kinds of beef and 6 kinds of chicken.

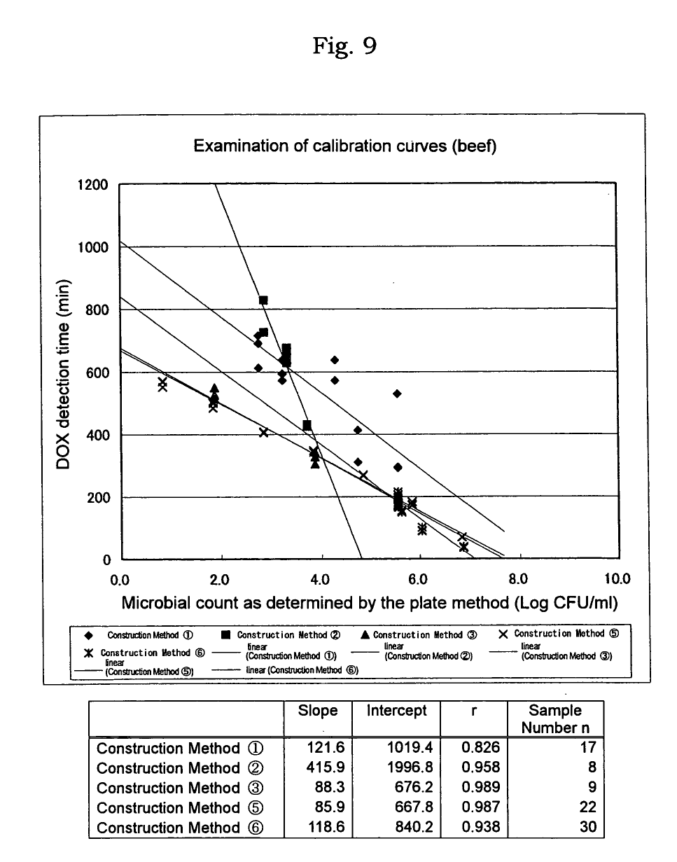

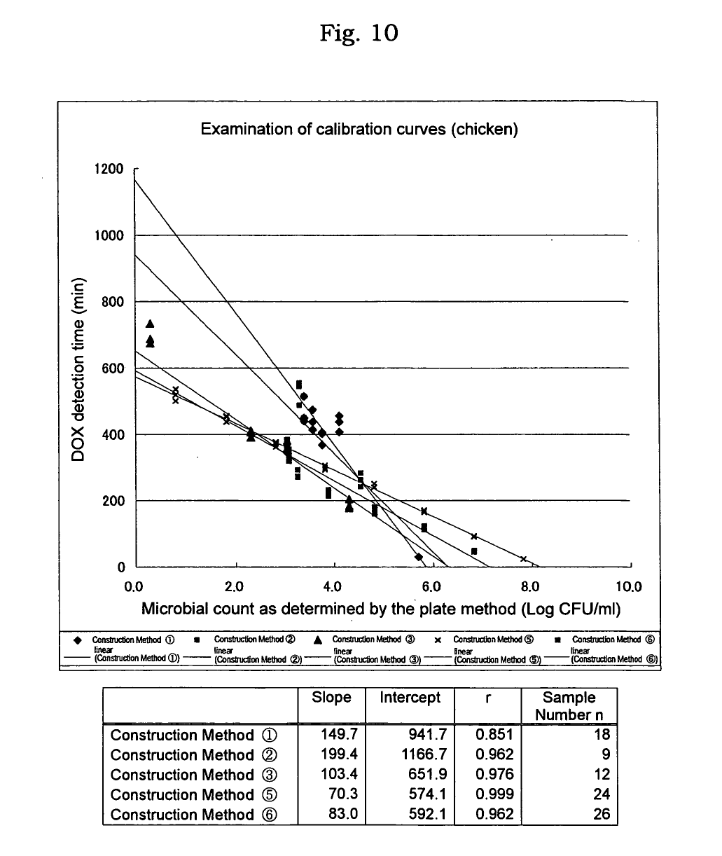

The measurement results were plotted, and regression curves were calculated by the least square method to provide calibration curves. The results obtained are shown in FIGS. 9 and 10.

Construction Method (II)

Construction Method (II) is a measurement according to the flowchart shown in FIG. 3, in which the specimen material is stored under predetermined conditions to increase or decrease the microbial count.

In the same manner as that in Construction Method (I), 10 g of a foodstuff and 90 ml of physiological saline were homogenized for one minute.

For DOX measurement, 1 ml of the homogenized liquid material and 1 ml of the special medium for DOX were injected into an electrode cell, and subjected to measurement. In order to determine quantitatively the microbial counts using the plate method, 1 ml of the homogenized liquid material obtained by the homogenization step, 1 ml of 10-fold dilution thereof, and 1 ml of 100-fold dilution thereof were respectively distributed into Petri dishes, and an agar medium was then poured. After culturing 48 hours at 35° C., the microbial counts were determined.

The remainder of the foodstuff left after the above measurement was stored for 24 and 48 hours at 4° C. and then subjected to the same operation as above. Since it is desirable that many samples in different ranges of concentration are subjected to measurement in order to obtain an accurate calibration curve, a sample having a somewhat increased microbial count compared to the sample before storage was obtained after 24 hours, and a sample having a significantly increased microbial count was obtained after 48 hours. A single accurate calibration curve is obtained from the measurement results at these three points.

The measurement results were plotted, and regression curves were calculated by the least square method to provide calibration curves. The results obtained are shown in FIGS. 9 and 10.

Construction method (III)

Construction Method (III) is a measurement according to the flowchart shown in FIG. 4, in which microbes are proliferated by the plate method using a liquid material obtained by homogenizing the specimen material, and the proliferated microbes are diluted as appropriate.

In the same manner as that in Construction Method (I), 10 g of a foodstuff and 90 ml of physiological saline were homogenized for one minute. On an agar plate, 0.1 ml of the homogenized liquid material thus obtained was smeared.

After 24 hours, microbes were picked from appropriate colonies on the agar plate using a platinum loop, and suspended in physiological saline.

The suspension was subjected to nine steps of 10-fold serial dilution. One ml of each dilution and 1 ml of the special medium for DOX were then injected into an electrode-equipped cell and subjected to DOX measurement.

Similarly, in order to determine quantitatively the microbial count by the plate method, 0.1 ml of a million-fold dilution of the suspension was smeared on an agar plate, and incubated for 48 hours at 35° C. before determining the microbial count. In this connection, in DOX measurement, 2 or 3 samples are measured for each concentration of the diluted suspensions, whereas in the measurement with the plate for quantitative determination of the microbial count, only one diluted suspension having the lowest concentration is measured, and the values for the rest are calculated using the dilution factors.

The measurement results were plotted, and regression curves were calculated by the least square method to provide calibration curves. The results obtained are shown in FIGS. 9 and 10.

Construction Method (V)

Construction Method (V) is a measurement according to the flowchart shown in FIG. 6, in which microbes are proliferated in DOX using a liquid material obtained by homogenizing the specimen material, and diluted as appropriate.

In the same manner as that in Construction Method (I), 10 g of a foodstuff and 90 ml of physiological saline were homogenized for one minute.

For DOX measurement, 1 ml of the homogenized liquid material and 1 ml of the special medium for DOX were injected into an electrode-equipped cell, and subjected to measurement.

After completion of the measurement, the solution in the electrode-equipped cell was subjected to 9 steps of 10-fold serial dilution. One ml of each dilution and 1 ml of a liquid medium were then injected into an electrode-equipped cell and subjected to DOX measurement.

Similarly, in order to determine quantitatively the microbial count by the plate method, 0.1 ml of a million-fold dilution of the solution in the electrode-equipped cell was smeared on an agar plate, and incubated for 48 hours at 35° C. before determining the microbial count.

In this connection, in DOX measurement, 2 or 3 samples are measured for each concentration of the diluted suspensions, whereas in the measurement with the plate for quantitative determination of the microbial count, only one diluted suspension having the lowest concentration is measured, and the values for the rest are calculated using the dilution factors.

The measurement results were plotted, and regression curves were calculated by the least square method to provide calibration curves. The results obtained are shown in FIGS. 9 and 10.

Construction Method (VI)

Construction Method (VI) is a measurement according to the flowchart shown in FIG. 7, in which the diluted material obtained by appropriate dilution in Construction Method (V) are added to a crushed homogenized liquid material of the specimen material.

In the same manner as that in Construction Method (I), 10 g of a foodstuff and 90 ml of physiological saline were homogenized for one minute.

For DOX measurement, 1 ml of the homogenized liquid material thus obtained and 1 ml of the special medium for DOX were injected into an electrode cell, and subjected to measurement.

After completion of the measurement, the solution in the electrode-equipped cell was subjected to 9 steps of 10-fold serial dilution. One ml of each dilution and 1 ml of a liquid medium were then injected into an electrode-equipped cell and subjected to DOX measurement.

Similarly, in order to determine quantitatively the microbial count by the plate method, 0.1 ml of a million-fold dilution of the solution in the electrode-equipped cell was smeared on an agar plate, and incubated for 48 hours at 35° C. before determining the microbial count.

In this connection, in DOX measurement, 2 or 3 samples are measured for each concentration of the sample liquids, whereas in the measurement with the plate for quantitative determination of the microbial count, only a sample liquid having the lowest concentration is measured, and the values for the rest are calculated using the dilution factors.

The measurement results were plotted, and regression curves were calculated by the least square method to provide calibration curves. The results obtained are shown in FIGS. 9 and 10.

From FIGS. 9 and 10 showing calibration curves obtained by each of the above methods, it is apparent that all methods result in a better correlation coefficient compared to the conventionally used Construction Method (I).

### EFFECTS OF THE INVENTION

The present invention is a method of simply and conveniently constructing a highly accurate calibration curve taking properties of the foodstuff into consideration. According to the present inventions, a method of constructing a calibration curve in a determination of microbial counts may be provided, and an immunoassay of the present invention is characterized in that samples of a specimen material are prepared so that microbial counts therein are altered.

1. A method of constructing a calibration curve in the course of a process for determining the microbial count in a specimen material, the method comprising the steps of:
1) preparing samples of the specimen material that have altered microbial counts in a number sufficient for constructing a calibration curve,
2) measuring a predetermined parameter for each of the samples obtained in Step 1, and, at the same time, determining quantitatively the microbial count in each of the corresponding samples, and
3) correlating the measured values for the predetermined parameter with the microbial counts measured by the quantitative determination, thereby constructing a calibration curve.

2. A method according to claim 1, wherein Step 1 comprises keeping the specimen material at ambient temperature or at a lower temperature to increase the microbial count, or freezing the specimen material to decrease the microbial count, thereby preparing the samples of the specimen material that have altered microbial counts.

3. A method according to claim 1, wherein Step 1 comprises homogenizing the specimen material to give a liquid material, proliferating the microbes in the liquid material, and diluting the proliferated microbes as appropriate, thereby preparing the samples of the specimen material that have altered microbial counts.

4. A method according to claim 1, wherein Step 1 comprises homogenizing the specimen material to give a liquid material, proliferating the microbes in the liquid material, diluting the proliferated microbes as appropriate, and then adding each of the diluted materials to a homogenized liquid material of the specimen material, thereby preparing the samples of the specimen material that have altered microbial counts.

5. A method according to any one of claims 1 to 4, wherein the microbial count is quantitatively determined by a standard plate count method in Step 2.

6. A method according to claim 1, wherein the predetermined parameter is measured using an apparatus for determining the microbial count in Step 2.

7. A method according to claim 6, wherein the apparatus for determining the microbial count comprises measuring the amount of dissolved oxygen using an oxygen electrode.

8. A method according to claim 7, wherein Step 1 comprises preparing the samples of the specimen material that have altered microbial counts by the apparatus which comprises measuring the amount of dissolved oxygen using an oxygen electrode.

9. An apparatus for determination of microbial counts comprising, or for conducting, a method according to claim 1.

10. An apparatus according to claim 9, wherein the apparatus comprises measuring the amount of dissolved oxygen using an oxygen electrode.

11. A process for determining microbial counts in a specimen material, which comprises a method according to claim 1.

12. An apparatus for determination of microbial counts comprising a process according to claim 11.
